(19) United States
(12) Patent Application Publication
(10) Pub. No.: US 2020/0367962 A1
(43) Pub. Date: Nov. 26, 2020

(54) MINIMALLY INVASIVE ASSEMBLY FOR LUNG ABLATION

(71) Applicant: Innoblative Designs, Inc., Chicago, IL (US)

(72) Inventor: Tyler Wanke, Chicago, IL (US)

(21) Appl. No.: 16/876,284

(22) Filed: May 18, 2020

Related U.S. Application Data
(60) Provisional application No. 62/850,327, filed on May 20, 2019.

(51) Int. Cl. A61B 18/14; A61B 34/30; A61B 18/16

(52) U.S. Cl. CPC A61B 18/1487; A61B 2018/00541; A61B 18/16; A61B 34/30

(57) ABSTRACT

The present disclosure is directed to a minimally invasive medical assembly including a delivery device in the form of a sheath configured to be delivered to a target site in a patient by being advanced through tortuous anatomy to deliver surgical and ablative devices to a target site (e.g. a lung tumor), to remove and ablate the target tissue. The invention allows for the delivery of various surgical and ablative devices to a target site without the need to surgically open and release the patient's chest cavity.

### CROSS-REFERENCE TO RELATED APPLICATIONS

The present application claims benefit of U.S. Provisional Application No. 62/850,327, filed May 20, 2019, the content of which is incorporated by reference herein in its entirety.

### FIELD OF THE INVENTION

The present disclosure relates generally to medical devices for ablating tissue, and, more particularly, to a minimally invasive medical assembly including a delivery device in the form of a guide sheath configured to be advanced through tortuous anatomy, particularly within a lung, and deliver surgical and ablative devices to a target site located within the lung tissue, wherein the surgical and ablative devices are configured to be deployed and controlled to access and ablate the target tissue.

### BACKGROUND

Ablation therapy is a type of minimally invasive procedure medical professionals (i.e., surgeons) use to destroy abnormal tissue that occurs with many conditions. For example, a doctor might use an ablation procedure to treat a cancerous tumor (i.e., completely destroy cancerous tissue) or to destroy (ablate) a small amount of tissue, such as in the heart so as to prevent and/or treat abnormal heart rhythms. In some instances, ablation therapy may be particularly difficult due to the location of the abnormal tissue to be treated and the degree of preciseness required to avoid ablating adjacent healthy tissue, such as when treating a tumor of the lung. For example, a lung tumor may be located deep within lung tissue. In order to access this tissue, invasive procedures would normally be required to surgically open up the lung, access the tumor, and then excise the tumorous tissue. Such procedures result in damage to otherwise healthy tissue, extended recovery times for the patient, and scarring and discomfort long after the procedure is completed.

### SUMMARY OF THE INVENTION

The present invention is directed to a minimally invasive medical assembly including a delivery device in the form of a sheath configured to be advanced through tortuous anatomy, particularly within a lung, and subsequently deliver surgical and ablative devices to a target site (e.g. a lung tumor), to remove and ablate the target tissue. Aspects of the invention utilize various surgical and ablative devices.

A first medical assembly comprises the sheath, having a proximal end and a distal end, with the distal end of the sheath configured to provide access to the target site. It is understood that the proximal end of the sheath may be accessible to a surgeon or other medical professional who can provide to the proximal end devices that are configured to be inserted into the sheath.

In an aspect of the invention a coring device is configured to be inserted into the proximal end of the sheath in order to access the distal end of the sheath. The coring device may be configured to surgically create a cavity in the target site. For example, the target site may be adjacent to a bronchiole in the lung and the coring device may be configured to surgically create an exit point in the bronchiole to access the target site adjacent to the bronchiole. In aspects of the invention, the first medical assembly may comprise a cutting device configured to surgically create an exit point in the bronchiole to provide access to the target site adjacent to the bronchiole. In aspects of the invention, once having access to the target site the coring device may be configured to surgically create a cavity at the target site, thereby removing some or all of the target tissue.

Aspects of the invention further comprise an extraction device configured to be inserted into the proximal end of the sheath and gain access to the distal end of the sheath. The extraction device may be configured to remove tissue from the cavity created by the coring device. The extraction device is configured to remove tissue from the distal of the sheath, through the sheath, to the proximal end of the sheath. From the proximal end of the sheath, the tissue may be removed and then further examined. The extraction device may be configured to use suction force to remove tissue from the cavity created by the coring device. In aspects of the invention, the sheath itself may act as the extraction device, for example by providing the suction force.

The medical assembly of the invention further comprises an ablation device configured to be inserted into the proximal end of the sheath and access the distal end of the sheath. The ablation device may be configured to enter the cavity created by the coring device and conduct and deliver energy for ablation to the target site. Various ablation devices may be utilized with the medical assembly.

In aspects of the invention the ablation device may comprise an inflatable member configured to transition from a collapsed configuration to an expanded configuration. The inflatable member has an interior surface and an exterior surface, and the inflatable member is configured to conduct and deliver the energy for ablation to the exterior surface of the inflatable member and to the target site when in the expanded configuration. The inflatable member may preferably be round or spherical, and may preferably be a balloon. The interior and/or exterior surface of the inflatable member may be elastic.

In aspects of the invention, the medical assembly may further comprising at least one lumen configured to extend from the proximal end of the sheath to the distal end of the sheath when the ablation device has accessed the distal end of the sheath. The medical assembly comprising at least one lumen may be configured to deliver a flued through the at least one lumen. In aspects of the invention, the ablation device may be configured to be in fluid communication with the at least one lumen to allow passage of a fluid from the at least one lumen to the ablation device.

For example, where the ablation device is an inflatable member, the ablation device may be configured to be in fluid communication with the at least one lumen to allow passage of a fluid from the at least one lumen to the interior surface of the inflatable member. The inflatable member may further comprise a plurality of perforations to allow the passage of a fluid from the interior surface of the inflatable member to the exterior surface of the inflatable member, and the ablation device may be configured to conduct the energy for ablation to be delivered by the fluid passing through the plurality of perforations to the target site when in the expanded configuration.

In some aspects of the invention, the inflatable device may further comprise one or more conductive wires disposed along at least a portion of an exterior surface of the inflatable member, the one or more conductive wires configured to conduct the energy for ablation to the target site.

In another aspect of the invention, the ablation device may comprise an expandable mesh assembly having a self-expanding mesh body configured to transition between a collapsed configuration and an expanded configuration. The mesh body may expand into a predefined shape. In aspects of the invention the predefined shape may be a round shape or a spherical shape. The mesh body of the invention comprises an electrically conductive material and is configured to conduct and deliver the energy for ablation to the target tissue when in the expanded configuration.

In aspects of the invention, the energy for ablation is monopolar energy. In other aspects of the invention the energy of ablation is bipolar. Where the energy for ablation is monopolar, the medical assembly may further comprise a monopolar return member configured to be placed on the exterior of the subject and the ablation device may be configured to conduct energy for ablation together with the monopolar return member. For example, the ablation may be configured to conduct energy for ablation from the ablation device through the target tissue in the direction of the monopolar return member. In aspects of the invention, the energy for ablation is radiofrequency (RF) energy.

Another medical assembly of the invention comprises a sheath having a proximal end and a distal end, the distal end of the sheath configured to provide access to the target site. The medical device comprises an ablation device configured to be inserted into the proximal end of the sheath and access the distal end of the sheath. The ablation device of the medical assembly comprises a first arm and second arm configured to be articulated to the target site. The first arm and second arm may be configured to apply pressure to a first point and a second point at the target site. For example, where the target site is a tumor, the first arm and second arm may be configured to apply pressure to opposing points on the tumor. The ablation device further configured to conduct and deliver energy for ablation from the first arm through the target site to the second arm. In aspects of the invention the energy for ablation is conducted and delivered when the arms apply pressure to the target site. The arms may apply pressure when the sheath is extended or advanced, causing the bipolar arms to become squeezed together.

The first arm and second arm may be connected at a central point or may be independent. In aspects of the invention, the energy for ablation is bipolar energy. In aspects of the invention, the energy for ablation is radiofrequency (RF) energy.

Advantageously, the present invention provides a minimally invasive medical assembly comprising a delivery device in the form of a sheath configured to provide surgical and ablative devices to a target site. In aspects of the invention, the sheath is advanced through tortuous anatomy, such as within the lung, and subsequently delivers surgical and ablative devices to a target site (e.g. a lung tumor), to remove and ablate the target tissue. In aspects of the invention, the sheath is configured to provide access to the target site by entering into and articulating along the interior of one or more vessels in the subject and in the target tissue to reach the target site.

Accordingly, aspects of the medical assembly comprise a controller that is configured to control articulation of the sheath. The controller may be configured to control the operation of one or more of the coring device, the cutting device, the extraction device, and the ablation device. In aspects of the invention, the controller may be a surgical robot.

Because the medical device is capable of delivering surgical and ablative devices directly to a target site through one or more vessels in a subject, the device is advantageous in treating lung tissue. For example, the one or more vessels through which the medical device delivers the surgical and ablative devices may be bronchioles. The sheath of the medical device may be configured to first enter the trachea of the subject and then articulated, for example by a controller, to the bronchioles and to the target site.

Advantageously, the target site may be a tumor and the medical assembly may be used to treat the tumor. The tumor may be a tumor of the lung and may be within the lung tissue.

It is understood that devices of the medical assembly of the present invention are shaped and sized to fit within and pass through the sheath of the assembly. The sheath is shaped and sized to fit within and pass through the one or more vessels (e.g., bronchial airway) through which in needs to pass to reach the target site (e.g. a lung tumor). The devices are configured to be deployable from the distal end of the sheath, Various devices may be designed to expand and become shaped and sized to apply at least a degree of contact upon the target site.

The assembly of the present invention allows for improved controlled over the ablation of tissue, particularly in normally isolated regions (i.e., tissue adjacent to tortuous pathways in the lung), allowing for application of RF energy in a controlled manner with little or no deleterious effect on surrounding healthy tissue or organs. Furthermore, the present invention allows for debulking of diseased tissue almost immediately by way of RF ablation, such that, upon treating the diseased tissue, necrosis of such tissue is immediate and the assembly can be completely removed once the procedure is completed, and thus does not present any issues that may be present with devices requiring implantation for a given period of time.

Aspects of the invention include methods of using the medical assemblies of the present invention. For example, aspects of the invention include a method for ablating a target tissue in a subject, comprising articulating a sheath comprising a proximal end and a distal end, within one or more vessels of the subject to reach a target site, and deploying one or more of the cutting device, the coring device, and the extraction device of the present invention; and thereafter deploying the ablation device to ablate the target tissue. In methods of the present invention, the target tissue is a tumor, for example a lung tumor, and the one or more vessels and vessels of the lung, such as bronchioles.

### BRIEF DESCRIPTION OF THE DRAWINGS

Features and advantages of the claimed subject matter will be apparent from the following detailed description of embodiments consistent therewith, which description should be considered with reference to the accompanying drawings, wherein:

FIG. 1 is an illustration of a medical assembly consistent with the present disclosure;

FIG. 2 is an illustration of a sheath of the medical assembly.

FIG. 3 in illustration of part of a controller consistent with the present disclosure;

FIG. 4 illustrates positioning and movement of the sheath through tortuous pathways within the bronchial airways of a lung, specifically illustrating the steerable nature of the sheath;

FIG. 5 illustrates deploying of a cutting device consistent with the present invention;

FIG. 6 illustrates deploying of a coring device consistent with the present invention;

FIG. 7 illustrates deployment of an inflatable ablation device consistent with the present invention.

FIG. 8 illustrates an ablation device of the present invention together with a monopolar return member;

FIG. 9 illustrates positioning of the an ablation device together with a monopolar return member in connection with a subject;

FIG. 10 illustrates deployment of a ablation device with a first arm and second arm consistent with the present invention;

FIG. 11 illustrates positioning of the first arm and the second arm of the ablation device to the target site.

FIG. 12 illustrates positioning of the first arm and second arm of the ablation device to apply pressure to the target site, the pressure applied by advancing the sheath of the medical assembly;

FIG. 13A-E illustrates positioning of the sheath at a bronchiole near A target site (FIG. 13A); deployment of an ablation device with a first arm and a second arm (FIG. 13B); positioning the first arm and the second arm of the ablation device adjacent to opposite sides of the target site (FIG. 13C); applying energy for ablation to the target site (FIG. 13D); and retracting or withdrawing the ablation device (FIG. 13E).

FIG. 14A-14C illustrates deployment of an extraction device consistent with the present invention to a target site (FIG. 14A); to extract tissue from the target site (FIG. 14B); to be transported from the distal end of the sheath to the proximal end of the sheath (FIG. 14C).

FIG. 15A-15G illustrate deployment of the inflatable ablation device into the cavity remaining from the illustration in FIG. 14A-C (FIG. 15A); the ablation device is configured into an expanded state (FIG. 15B); and energy for ablation is applied to the target site (FIG. 15C). The inflatable ablation device is then configured into the collapsed state (FIG. 15D) and partially retracted from the target site towards the bronchiole to a second site (FIG. 15E). The ablation device then conducts energy for ablation at the second site (FIG. 15F); and the ablation device and sheath are then retracted from the subject (FIG. 15G).

For a thorough understanding of the present disclosure, reference should be made to the following detailed description, including the appended claims, in connection with the above-described drawings. Although the present disclosure is described in connection with exemplary embodiments, the disclosure is not intended to be limited to the specific forms set forth herein. It is understood that various omissions and substitutions of equivalents are contemplated as circumstances may suggest or render expedient.

### DETAILED DESCRIPTION

The present disclosure is generally directed to a minimally invasive medical assembly including a delivery device in the form of a sheath configured to be delivered to a target site in a patient by being advanced through tortuous anatomy to deliver surgical and ablative devices to a target site (e.g. a lung tumor), to remove and ablate the target tissue. The invention allows for the delivery of various surgical and ablative devices to a target site without the need to surgically open and release the patient's chest cavity.

As will be described in greater detail herein, each of the devices of the present invention are configured to be advanced to the target site and transition between a delivery configuration, shaped and sized to fit within and pass through the sheath and thereby a pathway (i.e., bronchial airway), and a deployed configuration, in which the device is deployed from the distal end of the sheath and configured to perform its function. For example, with regard to the inflatable ablation device, in a deployed configuration the inflatable device is conjured to be expanded and is shaped and sized in such a manner to contact tissue at the target site and provide energy for ablation, such as non-ionizing radiation, such as radiofrequency (RF) energy, to treat the target tissue, specifically a diseased tissue, such as a cancerous tumor. For example, the inflatable ablation device may generally include an electrode array positioned along an external surface thereof, wherein the electrode array is configured to receive an electrical current from an energy source and conduct energy, the energy including RF energy. The parameters of the ablation, such elapsed time, total energy output, specific energy output pattern, etc., can be controlled and delivered based on the needs at the target site.

For ease of description and explanation, the following description focuses on the use of the articulating assembly within the tortuous passageways of the lung of a patient. However, it should be noted that the present invention can be used in any other organs which may include tortuous pathways, such as the heart or the vascular system.

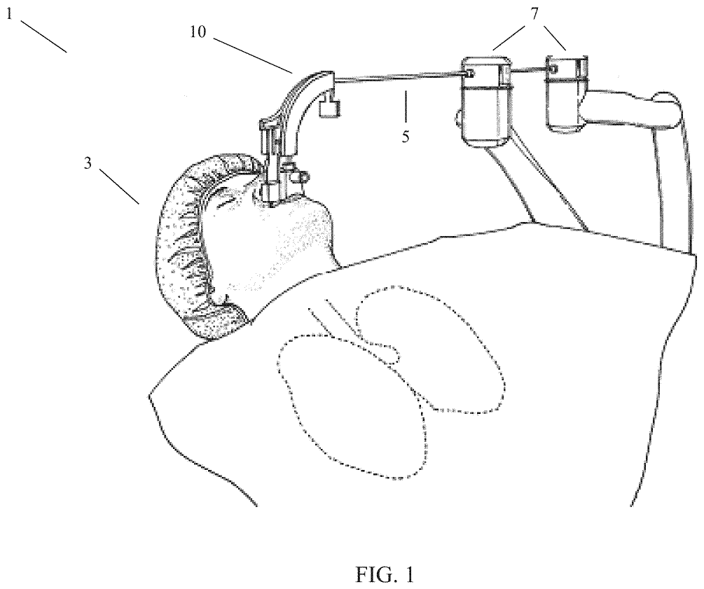

FIG. 1 is a schematic illustrations of a medical assembly 1 of the present invention for providing ablation of target tissue within the patient 3. The assembly system comprises a controller 10 placed at the mouth of the patient, and end effectors 3 that work together with the controller 10 to control articulation and advancement of the sheath 1 for delivery of the devices to the subject. The sheath 1 may be in the form of or provided as part of a scope or other access device configured to provide access to a target site within a patient 3, including, but not limited to, an endoscope, laparoscope, catheter, trocar, or other delivery device. Advantageously, the medical assembly 1 works with a surgical robot to control the articulation and advancement of the sheath 5 and any devices delivered to the distal end of the sheath 5.

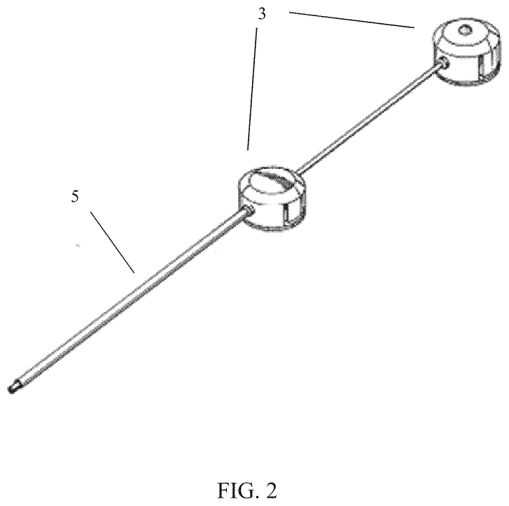

FIG. 2 is a view of the end effectors 3 of the medical assembly 1 used together with the controller to control articulation and advancement of the sheath 5. One end effector 3 can be used to rotate the sheath 5 and retract the sheath 5. The second end effector 3 can be used to advance and rotate the sheath 5. In aspects of the invention, the sheath 5 is connected to or comprises a pull wire used to position the sheath 5 through use of the end effectors. The sheath and devices may be steerable by way of a pull wire or tether that is anchored to a distal end of the sheath 5, thereby allowing maneuverability of the sheath 5 and devices as they move through the patient's vessels, such as bronchial airways. The pull wire or tether may be housed within a sheath housing. As generally understood, application of a pulling force upon the pull wire will generally result in a corresponding deflection of the distal end of the sheath 5, thereby allowing for the devices to be maneuvered past bifurcations or other obstructions. The medical assembly may also have a tetherless designed (i.e., the assembly does not include a pull wire or tether coupled to either of the first and second ablation devices).

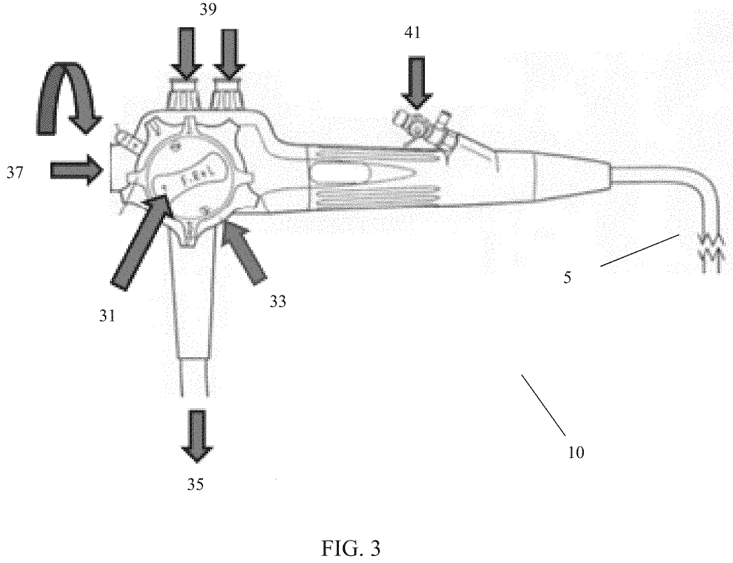

FIG. 3 is view of a portion of the controller 10 of the medical assembly 1. The controller 10 comprises a knob 31 that can lock the sheath 5 in a desired position. The knob 31 can be rotated 33 to unlock the sheath 5 to be articulated and advanced The controller 10 further comprises an RF generator 35 that provides the energy for ablation to the controller 10 to be delivered to the ablation device when at the distal end of the sheath 5. The controller 10 comprises an insert port 37 at the proximal end of the sheath 5 to allow for the insertion of surgical and ablative devices into the sheath 5 to the delivered to the distal end of the sheath. The controller 10 comprises a handle that can be used to rotate and orient devices that are inserted within the sheath. The control 10 further comprises saline ports 39 for providing fluid to one or more lumens of the medical assembly 1, for example saline fluid. In aspects of the invention, the medical assembly 1 and controller 10 further comprises a port 41 for injection a drug into the one or more lumens of the medical assembly 1 to be delivered to the distal end of the sheath 5 by one of the delivery devices, for example by the ablation device.

During an ablation treatment, the ablation device may be controlled by the controller 10 by determining output from the RF generator 35 to the ablation device. The RF generation 35 may generally provide RF energy (e.g., electrical energy in the radiofrequency (RF) range (e.g., 350-800 kHz). At the same time, saline may also be provided to the saline port 39 of the controller 10 and released through one or more lumens to the ablation device.

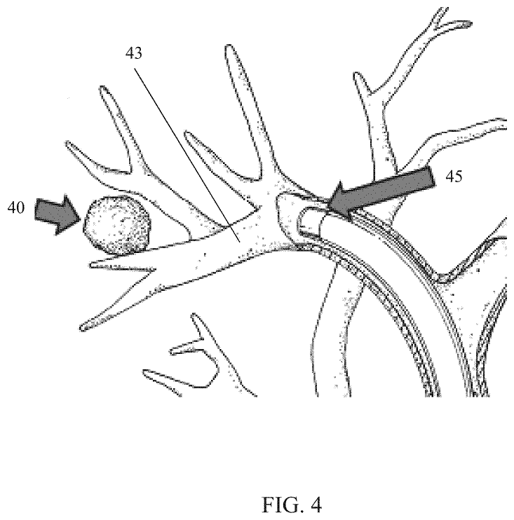

FIG. 4 shows a perspective view of one embodiment of the sheath 5 of the medical assembly 1 within the tissue of a patient 3. The sheath 5 is articulable through the bronchiole 43 of the patent to reach the target site 40 which comprises a lung tumor. The sheath 5 is configured to articulate and advance through the bronchiole 43 without damaging the bronchiole 43 or neighboring tissue. Although pictured is a bronchiole 43, the sheath 5 may be configured to articulate and advance through various vessels, such as vascular anatomy, of a patient without substantial damage to the vessel or neighboring tissue. The articulating assemblies may include a design similar to assemblies described in WO 2019/023328, the content of which is incorporated by reference herein in its entirety.

The sheath and various devices may further include sensors or the like to assist in providing visualization of the sheath to a surgeon during a procedure. Thus, in some embodiments, the sheath or devices inserted within the sheath may include a sensor, including, for example, an ultrasound transducer positioned along the sheath 5 adjacent to a distal end thereof or placed upon the body of the patient, wherein a line providing signal from the sensor may be housed within a sheath 5.

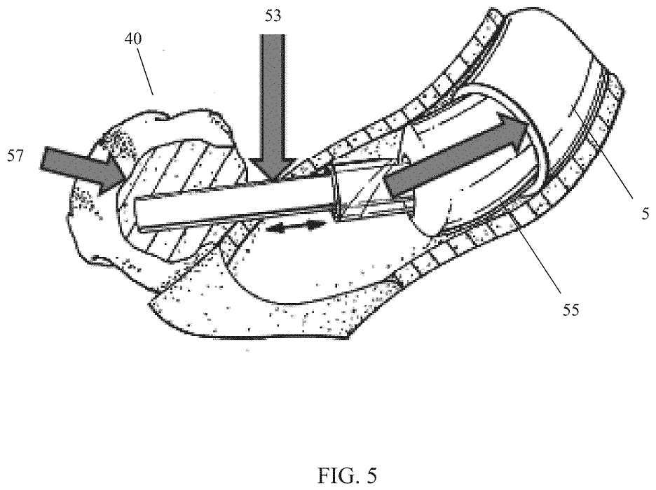

FIG. 5 shows a perspective view of a coring device 53 of the present invention. The coring device 53 is sized or shaped to fit within the sheath 5 and be delivered to the target site tumor 40. The coring device 53 or the sheath 5 itself may further comprise an inner guide sheath 55 which is sized and shaped to fit within the sheath 5 and be in communication with the coring device 53 or other delivery devices. The guide sheath 55 may control operation of the device such as the coring device 53. For example, the guide sheath 55 may be advanced or withdrawn to move the coring device 53 back and forth in a saw-like motion to create an exit point in the vessel of the patient and to remove a portion of the target site tumor 40, creating a cavity in the tissue 57, for example by removing the core of the tumor 40 to make room for the ablation device. An extraction device may also be delivered to the distal end of the sheath prior to insertion of the coring device to extract, for example by applying suction, to remove the core of the tumor 40 for biopsy. In aspects of the invention, the sheath 5 itself or guide sheath 55 may alternatively provide suction to remove the core of the tumor.

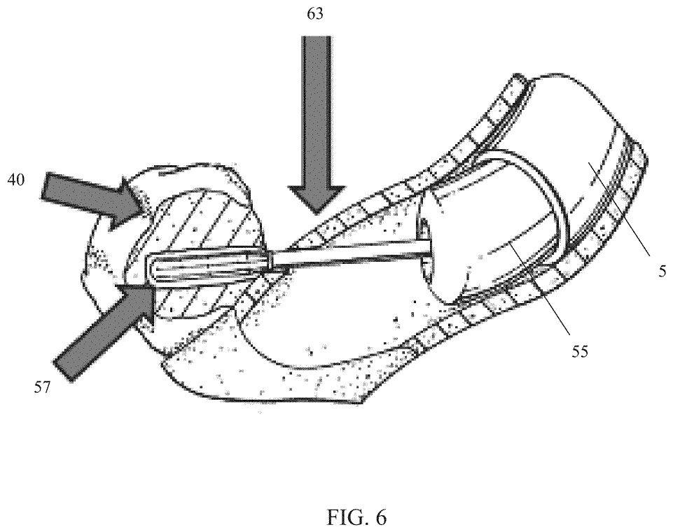

FIG. 6 shows a perspective view of an inflatable ablation device of the present invention in a collapsed configuration 63. The collapsed ablation device 63 is sized or shaped to fit within the sheath 5 delivered to the distal tip of the sheath 5. As shown, the inflatable ablation device includes a flexible body configured to have a collapsed configuration 63 and also entirely fit within the guide sheath 55, such that, upon advancing the guide sheath 55 to the target site, the ablation device can be extended from the distal end of the guide sheath 55 and the surgeon can control transitioning of the inflatable ablation device from a delivery configuration to a deployed configuration. The collapsed inflatable device 63 is further configured to enter the cavity 57 in the tumor 40 creating by the coring device 53.

In aspects of the invention, the inflatable ablation device may include an inner balloon (not shown) configured to receive a fluid or gas coupled to a source controllable via a controller 10, such as a valve or the like. Upon inflation of the inner balloon, the inflatable ablation device may correspondingly expand to assume the deployed configuration. In inflatable ablation device may be further coupled to one or more lumens of the sheath to provide a fluid line, in which the flow of conductive fluid from the controller 10 to the ablation device may be controlled manually via a valve or the like. Furthermore, the electrical line (not shown) coupling the RF generator 35 to an ablation device may further be housed within a separate sheath (not shown). The inflatable ablation device may include a design as described in U.S. Patent Publication No. 2016/0317221, the content of which is incorporated by reference herein in its entirety.

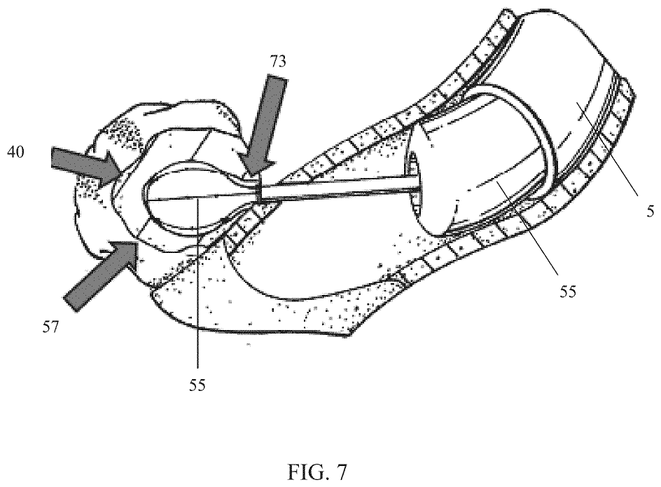

FIG. 7 shows a perspective view of the ablation device in a partially expanded configuration 73. The ablation device is configured to expand 73 within the cavity 57 of the tumor 40 created by the coring device. In another aspect of the invention, rather than an inflatable balloon, the ablation device 73 may comprise an expandable mesh assembly having a self-expanding mesh body configured to transition between a collapsed configuration and an expanded configuration. The mesh body may expand into a predefined shape, such as the same shape intended by the inflatable ablation device. In aspects of the invention the predefined shape may be a round shape or a spherical shape. The mesh body of the invention comprises an electrically conductive material and is configured to conduct and deliver the energy for ablation to the target tissue when in the expanded configuration.

The ablation device 73 may include an electrode array is composed of a plurality of conductive members (e.g., conductive wires) 55. In some embodiments, each of the plurality of conductive wires 55, or one or more sets of a combination of conductive wires 55, is configured to independently receive an electrical current from the RF generator 35 and independently conduct energy, the energy including RF energy. This allows energy to be selectively delivered to a designated conductive wire or combination of conductive wires. This design also enables the ablation device 73 to function in a bipolar mode because a first conductive wire (or combination of conductive wires) can deliver energy to the surrounding tissue through its electrical connection with the RF generator while a second conductive wire (or combination of conductive wiress) can function as a ground or neutral conductive member. It is understood that where a mesh body is used, the body of the ablation device itself may compose the combination of conductive wires.

In some embodiments, the ablation device 73 is configurd to provide RF ablation via a virtual electrode arrangement, which includes distribution of a fluid along an exterior surface of the ablation device and, upon activation of the electrode array, the fluid may carry, or otherwise promote, energy emitted from the electrode array to the surrounding tissue. For example, as previously described, the ablation device includes an interior surface configured to receive the conductive fluid therein from the fluid source through one or more lumens of the sheap. The ablation device may include a plurality of ports, perforations, or apertures configured to allow the fluid to pass therethrough, or weep, from the interior surface to the external surface of the ablation device. Accordingly, upon positioning the ablation device at a target site and subsequently transitioning the device from a collapsed configuation 63 to an expanded configuration 73, the electrode array can be activated and fluid can be delivered to the internal surface of the ablation device. The fluid weeping through the perforations to the outer surface of the ablation device is able to carry energy from electrode array, thereby creating a virtual electrode. Accordingly, upon the fluid weeping through the perforations, a pool or thin film of fluid is formed on the exterior surface of the ablation device and is configured to ablate surrounding tissue via the RF energy carried from the electrode array.

Advantageously, the ablation devices of the present invention provide uniform ablation depth into the target site, for example uniform ablation depth of the target tumor 40 to avoid ablation of surrounding healthy tissue.

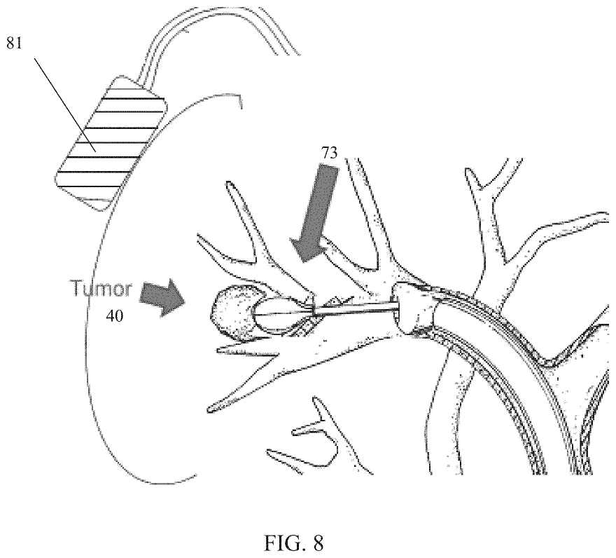

FIG. 8 and FIG. 9 show perspective views of the ablation device 73 together with a monopolar return member, specifically a monopolar return pad 81. Where the energy for ablation is monopolar, the medical assembly 1 may be configured to be placed on the exterior of the subject and the ablation device may be configured to conduct energy for ablation together with the monopolar return member 81. For example, the ablation may be configured to conduct energy for ablation from the ablation device 73 through the target site tumor 40 in the direction of the monopolar return member. In aspects of the invention, the energy for ablation is radiofrequency (RF) energy. FIG. 9 shows the medical assembly comprising a monopolar return pad 81 as shown in a perspective view with the patient 5. As pictured, the monopolar return pad is paced on the exterior chest of the patient 5 and the ablation device 57 is delivered to the target site tumor 40 by the sheath 5 as controlled by the controller 10. The ablation device 57 is on the opposite side of the tumor 40 from the monopolar return pad 81 and energy for ablation is directed through the tumor 40 towards the monopolar return pad.

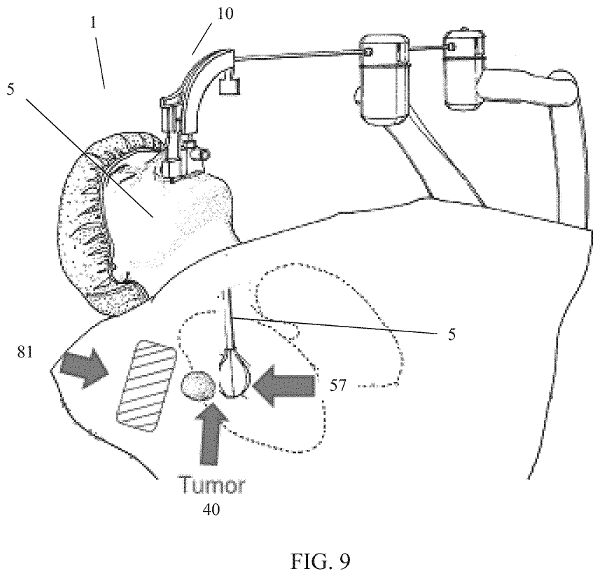

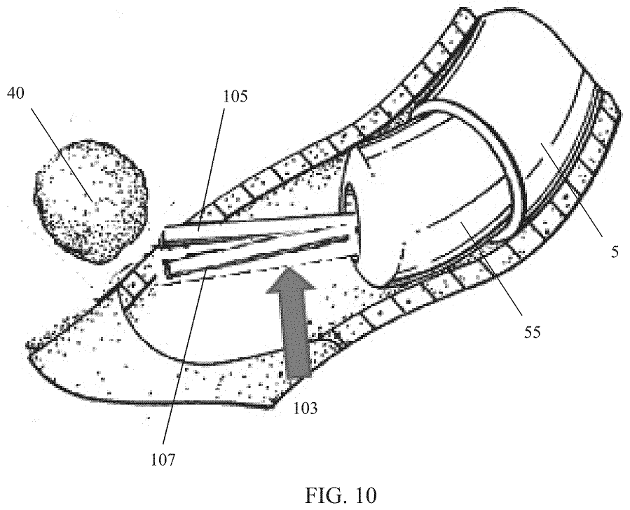

FIG. 10 shows a perspective view of an ablation device 103 of the present invention that uses a first arm 105 and a second arm 107. The two-arm ablation device 103 is sized or shaped to fit within the sheath 5 and be delivered to the target site tumor 40. The two-arm ablation device 103 or the sheath 5 itself may further comprise an inner guide sheath 55 which is sized and shaped to fit within the sheath 5 and be in communication with the two-arm ablation device 103. The guide sheath 55 may control operation of the device such as the two-arm ablation device 103. For example, once the two-arm device is deployed from the sheath, by moving the guide sheath 55 forward the two arms may be contracted together as they are pulled back into the sheath 5. By retracting the guide sheath 55, the first arm 105 and second arm 107 may spread apart.

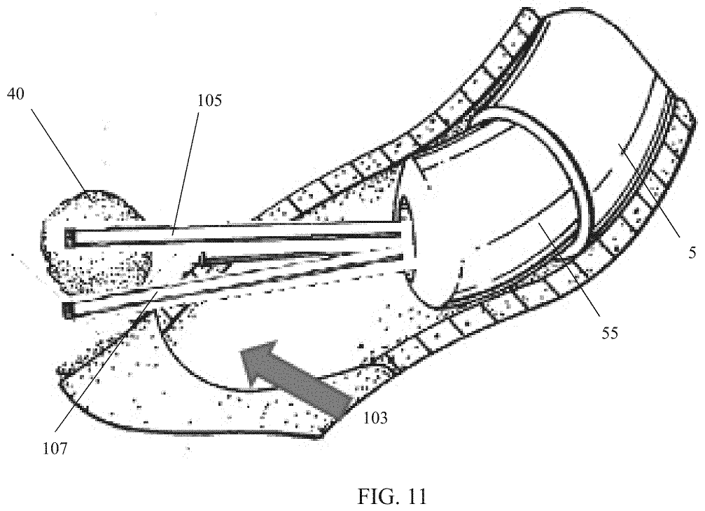

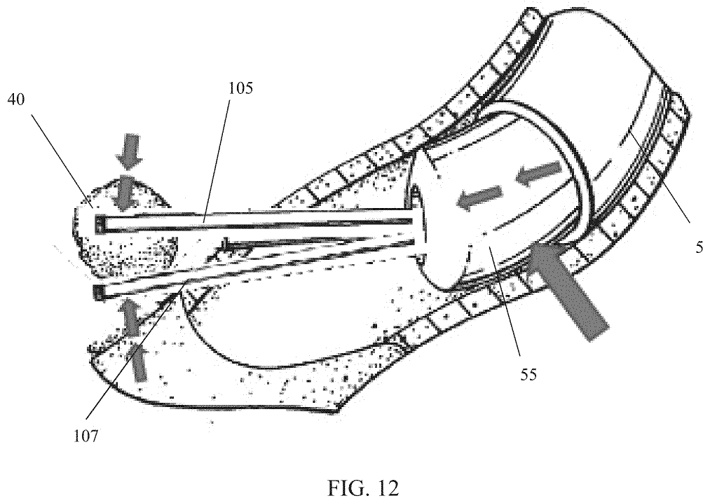

FIG. 11 and FIG. 12 show perspective views of the two-arm ablation device 103, showing the first arm 105 and the second arm 107 articulated and positioned to a first point and a second point on the target site tumor 40. As shown in FIG. 12, by moving the guide sheath 5 forward the first arm 105 and second arm 107 become contracted together applying pressure to the opposite sides of the tumor 40. Once pressure is applied, the two-arm ablation device is configured to conduct and deliver energy for ablation from the first arm 105 through the tumor 40 to the second arm 107. The first arm 105 and second arm 107 may be connected at a central point (not pictured) or may be independent. In aspects of the invention, the energy for ablation is bipolar energy. Advantageously, the two-arm ablation assembly does not require the use of a coring device or evacuation device, which can carry the risk of transporting tumorous tissue to adjacent or distant parts of the patient, facilitating unintentional deposition of the tumor tissue. Moreover, because energy for ablation is conducted and delivered from the first arm 105 to the second arm 107 directly through the tumor, ablation of adjacent healthy tissue is minimized.

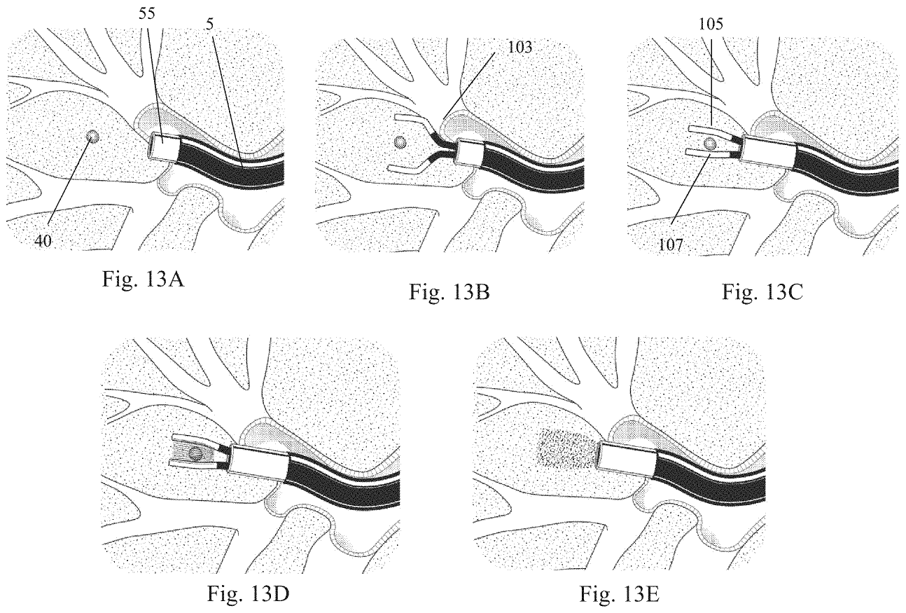

FIG. 13A-E show perspective views of a method using the two-arm ablation device 103. A sheath 5 comprising a guide sheath 55 is deployed through the bronchiole of a subject to a point adjacent to a target site and the guide sheath 55 extended and the ablation device 63 deployed to the target site in a collapsed state (FIG. 13A). The two-arm ablation device 103 is deployed from the guide sheath 55 and articulated towards the tumor 40 (FIG. 13B). The guide sheath 55 is then advanced forward contracting the first arm 105 and the second arm 107 of the two-arm device 103 to a first point and a second point on opposite sides of the tumor 40 (FIG. 13C). Bipolar RF energy is then conducted and delivered from the first arm 105 through the tumor 40 to the second arm 107, ablating the tumorous tissue 40, but causing only minimal damage to neighboring tissue (FIG. 13D). The two-arm ablation device 103 is then retracted into the sheath 5 to be removed from the patient (FIG. 13E).

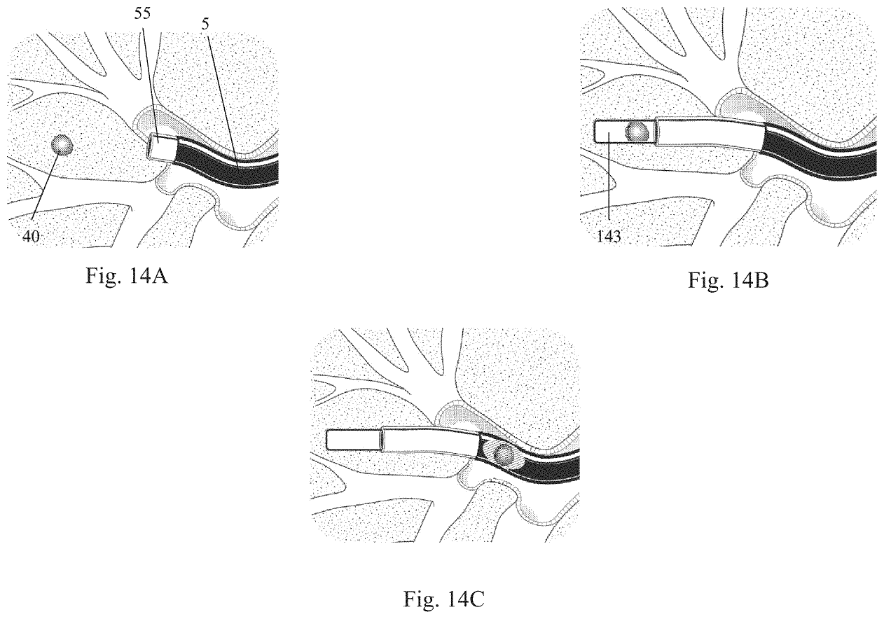

FIG. 14A-C show perspective views of a method of using an extraction device of the invention to remove tumorous tissue. A sheath 5 comprising a guide sheath 55 is deployed through the bronchiole of a subject to appoint adjacent to a target site tumor 40 (FIG. 14A). The extraction device 143 is deployed from the guide sheath 55 and articulated towards the tumor 40 to envelop the tumor (FIG. 14B). Suction is then provided through the sheath 5 and/or guide sheath 55 to extract the tumor 40 to the proximal end of the sheath 5 to be analyzed or disposed of. Because the tumor 40 was enveloped by the extraction device 143 prior to extraction, any tissue from the tumor 40 that may debris during extraction will remain within the medical assembly 1, and will not deposit in neighboring or distant tissue in the patient.

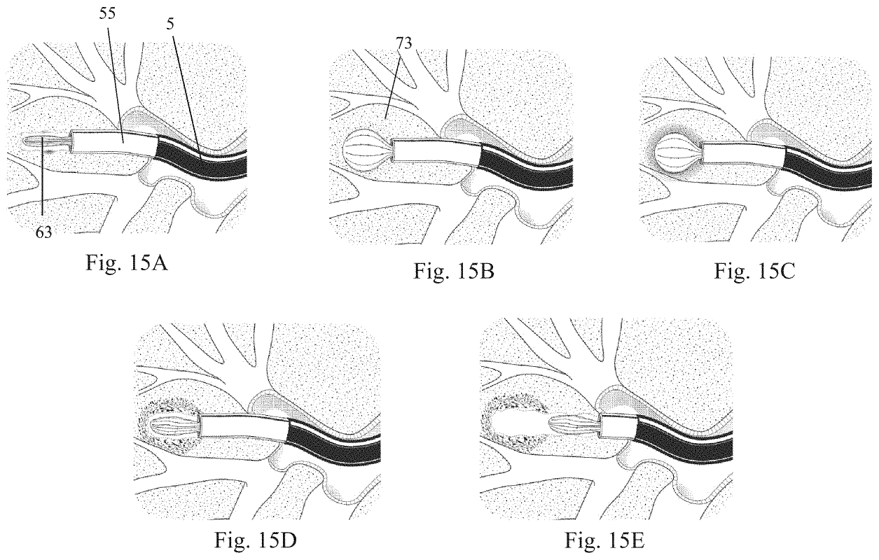

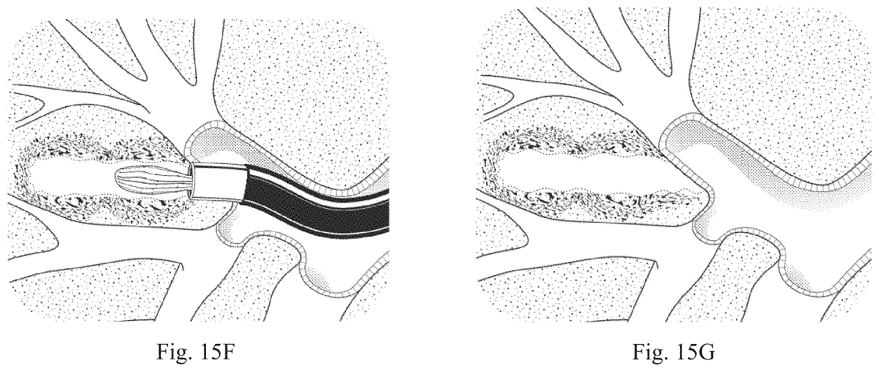

FIG. 15A-G show perspective views of a method of using an inflatable balloon or mesh body ablation device of the invention. A sheath 5 comprising a guide sheath 55 is deployed through the bronchiole of a subject to a point adjacent to a target site tumor 40 (FIG. 13A). The target site may be the site of a tumor. Prior to deployment of the ablation device 63, a cutting device may have created an exit point in the vessel (e.g. bronchiole) of the patient, and a coring device may be have been deployed to create a cavity in the tumor and/or an extraction device may have been deployed to remove part or all of the tumor. The ablation device is then adapted into an expanded state 73, preferably contacting the adjacent tissue (FIG. 15B). Energy for ablation is then applied at a fixed distance into the adjacent tissue, ablating targeted tissue while minimizing damage to healthy tissue (FIG. 15C). The ablation device is then adapted into a contracted state 63 (FIG. 15D). The guide sheath 55 and ablation device 63 are then partially retracted towards the sheath to a second point (FIG. 15E). Energy for ablation is once again applied at a fixed distance into the adjacent tissue (FIG. 15F). Optionally the ablation device may be adapted back into an expanded configuration prior to applying energy for ablation. The advantage of applying energy for ablation at the second point closer to the sheath, is that any tumorous tissue that was deposited by the guide sheath, coring device, or extraction device will be ablated, preventing the deposited tumorous tissue from surviving. Finally, the ablation device 63 and the sheath 5 are retracted from the subject.

In some embodiments, the ablation devices of the present invention may be further configurd to provide RF ablation via a virtual electrode arrangement, which includes distribution of a fluid along an exterior surface of the ablation device. Upon activation of the ablation device, the fluid may carry, or otherwise promote, energy emitted from the electrode array to the surrounding tissue. For example, the inflatable ablation device or mesh assembly may include a plurality of ports or apertures configured to allow the fluid to pass there through, or weep, from an interior surface of the device to an external surface of the device. Accordingly, the fluid may be a conductive fluid (e.g., saline).

The medical assembly may further include an imaging modality. For example, during a procedure, the imaging modality may provide an operator (e.g., surgeon) with a visual depiction of distal end of the delivery device during advancement towards the target site and may further provide visual depiction of the ablation devices during delivery and deployment thereof when positioning for subsequent ablation of target tissue. For example, in some embodiments, the imaging modality may be configured to receive sensing input from the delivery device (i.e. the sheath), the cutting device, the coring device, the extraction device, and the ablation devices (e.g., sensors on the scope or the devices, such as ultrasound, video, images, etc.) so as to provide an accurate display to the surgeon during a procedure. The imaging modality may provide a medical imaging procedure, including, but not limited to, ultrasound (US), wavelength detection, X-ray-based imaging, illumination, computed tomography (CT), radiography, and fluoroscopy, or a combination thereof, such that, when viewed under such a medical imaging procedure provided by the imaging modality, the visibility of the target site is enhanced and a surgeon can better maneuver the scope and each of the devices during a procedure.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications and further embodiments are possible beyond what is shown and described herein. The subject matter herein contains information, exemplification, and guidance that can be adapted to create various other embodiments.

What is claimed is:

1. A medical assembly for ablating a target site in the tissue of a subject, comprising
a sheath having a proximal end and a distal end, the distal end of the sheath configured to provide access to the target site;
a coring device configured to be inserted into the proximal end of the sheath and access the distal end of the sheath, the coring device configured to surgically create a cavity in the target site;
an extraction device configured to be inserted into the proximal end of the sheath and access the distal end of the sheath, the extraction device configured to remove tissue from the cavity created by the coring device; and
an ablation device configured to be inserted into the proximal end of the sheath and access the distal end of the sheath, the ablation device configured to enter the cavity created by the coring device and conduct and deliver energy for ablation to the target site.

2. The medical assembly of claim 1, wherein the extraction device is configured to use suction force to remove the tissue from the cavity created by the coring device.

3. The medical assembly of claim 1, wherein the ablation device comprises:
an inflatable member configured to transition from a collapsed configuration to an expanded configuration, the inflatable member having an interior surface and an exterior surface, the inflatable member configured to conduct and deliver the energy for ablation to the exterior surface of the inflatable member and to the target site when in the expanded configuration.

4. The medical assembly of claim 3, further comprising at least one lumen configured to extend from the proximal end of the sheath to the distal end of the sheath when the ablation device has accessed the distal end of the sheath, wherein:
the inflatable member is configured to be in fluid communication with the at least one lumen to allow passage of a fluid from the at least one lumen to the interior surface of the inflatable member,
the inflatable member comprises a plurality of perforations to allow the passage of a fluid from the interior surface of the inflatable member to the exterior surface of the inflatable member; and
the ablation device is configured to conduct the energy for ablation to be delivered by the fluid passing through the plurality of perforations to the target site when in the expanded configuration.

5. The medical assembly of claim 3, further comprising one or more conductive wires disposed along at least a portion of an exterior surface of the inflatable member, the one or more conductive wires configured to conduct the energy for ablation to the target site.

6. The medical assembly of claim 3, wherein the energy for ablation is monopolar energy.

7. The medical assembly of claim 6, wherein the medical assembly further comprises a monopolar return member configured to be placed on the exterior of the subject and the ablation device is configured to conduct energy for ablation from the exterior surface of the inflatable member through the target tissue in the direction of the monopolar return member.

8. The medical assembly of claim 1, wherein the ablation device comprises:
an expandable mesh assembly having a self-expanding mesh body configured to transition between a collapsed configuration and an expanded configuration, in which the mesh body expands into a predefined shape, the mesh body comprising an electrically conductive material and configured to conduct and deliver the energy for ablation to the target tissue when in the expanded configuration.

9. The medical assembly of claim 1, wherein the sheath is configured to provide access to the target site by entering into and articulating along the interior of one or more vessels in the target tissue to reach the target site.

10. The medical assembly of claim 9, further comprising a controller that is configured to control articulation of the sheath.

11. The medical assembly of claim 9, wherein the controller is configured to control the operation of one or more of the coring device, the extraction device, and the ablation device.

12. The medical assembly of claim 11, wherein the controller is a surgical robot.

13. The medical assembly of claim 9, wherein the target site is lung tissue.

14. The medical assembly of claim 13, wherein the one or more vessels are bronchioles.

15. The medical assembly of claim 14, wherein the channel is configured to be inserted into the trachea and articulated to reach the interior of the bronchioles.

16. The medical assembly of claim 1, wherein the target site is a tumor.

17. A medical assembly for ablating a target site in the tissue of a subject, comprising
a sheath having a proximal end and a distal end, the distal end of the sheath configured to provide access to the target site;
an ablation device configured to be inserted into the proximal end of the sheath and access the distal end of the sheath, the ablation device comprising a first arm and second arm configured to be articulated to and apply pressure to a first point and a second point at the target site, the ablation device further configured to conduct and deliver energy for ablation from the first arm through the target site to the second arm when the pressure is applied.

18. The medical assembly of claim 17, wherein the energy for ablation is bipolar energy.

19. The medical assembly of claim 17, wherein the first arm and second arm are connected at a central point.

20. The medical assembly of claim 17, wherein the sheath is configured to provide access to the target site by entering into and articulating along the interior of one or more vessels in the target tissue to reach the target site.

21. The medical assembly of claim 22, wherein articulation of the sheath is used to articulate the first arm and the second arm to apply pressure to the first point and the second point at the target site.
